(19) United States
(12) Patent Application Publication
(10) Pub. No.: US 2025/0090160 A1
(43) Pub. Date: Mar. 20, 2025

(54) SUTURE ANCHOR DEVICE, SYSTEM AND METHOD OF USE

(71) Applicant: AEVUMED, INC., Malvern, PA (US)

(72) Inventors: Miles O. Curtis, Philadelphia, PA (US); Saif Khalil, Malvern, PA (US)

(21) Appl. No.: 18/886,054

(22) Filed: Sep. 16, 2024

Related U.S. Application Data
(60) Provisional application No. 63/582,975, filed on Sep. 15, 2023.

(51) Int. Cl. A61B 17/04

(52) U.S. Cl. CPC A61B 17/0401; A61B 2017/0409; A61B 2017/0412; A61B 2017/0414; A61B 2017/044

(57) ABSTRACT

A suture anchor system includes a suture anchor and an eyelet shuttle. The suture anchor includes an elongate anchor body having at least one anchoring element, and multiple openings in a proximal face of the elongate anchor body. The eyelet shuttle includes an elongate shuttle body having an eyelet and at least one eyelet lock distal of a proximal threaded portion, and a proximal shuttle opening in a proximal face of the elongate shuttle body.

### CROSS-REFERENCE TO RELATED APPLICATIONS

This application claims priority to U.S. provisional application No. 63/582,975 filed on Sep. 15, 2023 incorporated herein by reference in its entirety.

### BACKGROUND OF THE INVENTION

Knotless suture anchors are used in various surgical procedures including orthopedic and soft tissue surgeries to anchor and secure soft tissue (such as tendons or ligaments) to bone. These anchors provide a secure fixation point for sutures, which are used to reattach or repair damaged tissue. Knotless suture anchors are commonly used in procedures like rotator cuff repairs, labral repairs in the shoulder, Achilles tendon repairs, and ligament reconstructions.

Knotless suture anchors can be designed with mechanisms that allow for the controlled tensioning and secure fixation of sutures, which are important aspects that contribute to the success of the surgical repair. Tension control and securement ensures that the repaired tissue is appropriately aligned and tensioned. This is critical for optimal tissue healing, as well as for restoring the tissue's functionality and biomechanics. Incorrect tension can otherwise lead to tissue necrosis, impaired blood flow, delayed healing, and suboptimal functional outcomes. Adequate tensioning of sutures also helps distribute mechanical loads evenly across the repaired tissue, bone, and surrounding structures. In procedures involving joints, such as ligament reconstructions, proper tension is important for restoring joint stability and maintaining a natural range of motion. Proper locking of the suture to anchor further minimizes the risk of suture slippage, loosening, or dislodgement, which could lead to surgical failure and the need for revision procedures. These factors ultimately contribute to the long-term durability of the surgical repair, enable the repaired tissue to withstand physiological loads and stresses, reduce the likelihood of re-injury, and enhance overall patient comfort, satisfaction and outcomes.

Accordingly, devices, system and methods that can offer an enhanced ability to control suture tension and securement will provide a significant advancement to the art. Embodiments described herein fit this need.

### SUMMARY OF THE INVENTION

In one embodiment, a suture anchor system includes a suture anchor having an elongate anchor body having at least one anchoring element, and a plurality of openings in a proximal face of the elongate anchor body; and an eyelet shuttle having: an elongate shuttle body having an eyelet and at least one eyelet lock distal of a proximal threaded portion, and a proximal shuttle opening in a proximal face of the elongate shuttle body. In one embodiment, the plurality of openings comprises a central opening and at least one driver opening. In one embodiment, the at least one driver opening is one of a pair of driver openings. In one embodiment, the pair of driver openings are configured on opposing sides of the central opening. In one embodiment, the central opening is configured between the pair of driver openings. In one embodiment, the central opening is configured along a central axis of the elongate anchor body. In one embodiment, the plurality of openings comprises at least one eyelet lock opening. In one embodiment, the eyelet lock opening is one of a pair of eyelet lock openings. In one embodiment, the pair of eyelet lock openings are configured on opposing sides of the central opening. In one embodiment, a central opening is configured between the pair of eyelet lock openings. In one embodiment, the central opening includes at least one internal slot running parallel to the central axis. In one embodiment, the at least one internal slot is one of a pair of internal slots running parallel to the central axis. In one embodiment, the pair of internal slots are configured on opposing sides of the central opening. In one embodiment, the central opening is configured between the pair of internal slots and along with the pair of eyelet lock openings define a common proximal void. In one embodiment, the common proximal void comprises a perimeter having at least four convex regions. In one embodiment, the distal end comprises a pointed distal tip. In one embodiment, a distal eyelet is configured between the distal tip and a first distal barb. In one embodiment, the at least one anchoring element comprises a plurality of barbs. In one embodiment, each of the plurality of barbs tapers away from a first diameter to a second diameter. In one embodiment, the system includes a proximal barb comprising a plurality of recesses. In one embodiment, the plurality of recesses is four recesses equally spaced apart. In one embodiment, the proximal barb tapers away from a first diameter to a third diameter, wherein the third diameter is greater than the second diameter. In one embodiment, the at least one eyelet lock is configured to arrest rotational movement of the eyelet while the threaded portion is rotating to separate the eyelet and threaded portion. In one embodiment, the at least one eyelet lock is configured to allow axial movement of the eyelet within the anchor while the threaded portion is rotating and while the eyelet and threaded portion are separated. In one embodiment, the at least one eyelet lock is one of a pair of eyelet locks. In one embodiment, the pair of eyelet locks are a set of opposing protrusions. In one embodiment, the system includes an inner driver having an elongate inner driver body and an inner driver distal portion configured to interface with the shuttle opening; and an outer driver having an elongate outer driver body an outer driver distal tip configured to interface with a pair of driver openings in a proximal face of the elongate anchor body, the elongate outer driver body configured to accept insertion of the elongate inner driver body at least partially therethrough. In one embodiment, the outer driver distal tip comprises a pair of prongs configured to interface with a pair of driver openings. In one embodiment, the inner driver distal portion has a non-circular cross-sectional profile portion. In one embodiment, the inner driver distal portion has a non-circular cross-sectional profile portion that terminates into a circular distal tip. In one embodiment, the system includes a wireloop tab comprising a wireloop connected to a tap having at least one surface configured to interface with a surface of the outer driver.

In one embodiment, a method for inserting a suture anchor includes the steps of loading an anchor and an eyelet shuttle onto an outer driver, the eyelet shuttle comprising an eyelet distal of a proximal threaded portion; passing at least one suture thought the eyelet shuttle; driving the anchor into bone by advancing the outer driver towards the bone; advancing an inner driver into the outer driver; and rotating the inner driver to separate the eyelet and the proximal threaded portion. In one embodiment, rotating the inner driver moves the eyelet axially within the anchor. In one embodiment, rotating the inner driver in a first direction locks the at least one suture. In one embodiment, rotating the inner driver in a second direction opposite the first direction unlocks the at least one suture.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIGS. 1A and 1B are alternate perspective views of a suture anchor according to one embodiment.

FIGS. 2A and 2B are alternate perspective views of an eyelet shuttle according to one embodiment, and FIGS. 2C and 2D show a functional cutaway view of the eyelet shuttle within an anchor in an unlocked position (FIG. 2C) and a locked position (FIG. 2D), locking the suture in place according to one embodiment.

FIG. 3A is a side view of an inner driver, FIG. 3B is a side view of an outer driver, FIG. 3C is a perspective magnified view of a distal portion of an outer driver, FIG. 3D is a perspective magnified view of a distal portion of an inner driver, and FIG. 3E is a perspective magnified view of a distal portion of a driver assembly, all according to one embodiment.

FIG. 4 is a diagram of suture anchor kit components according to one embodiment.

FIG. 5 is a flow chart of a method for inserting a suture anchor according to one embodiment.

FIGS. 6A through 6H are illustrations of a method for implanting a suture anchor according to one embodiment.

### DETAILED DESCRIPTION OF THE INVENTION

It is to be understood that the figures and descriptions of the present invention have been simplified to illustrate elements that are relevant for a more clear comprehension of the present invention, while eliminating, for the purpose of clarity, many other elements found in suture anchor devices, systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Ranges: throughout this disclosure, various aspects of the invention can be presented in a range format. It should be understood that the description in range format is merely for convenience and brevity and should not be construed as an inflexible limitation on the scope of the invention. Where appropriate, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Referring now in detail to the drawings, in which like reference numerals indicate like parts or elements throughout the several views, in various embodiments, presented herein is a suture anchor device, system and method.

Embodiments of a suture anchor device, system and method described herein, including components such as an eyelet shuttle and a driver assembly having an inner and outer driver, enable enhanced suture tension control and securement.

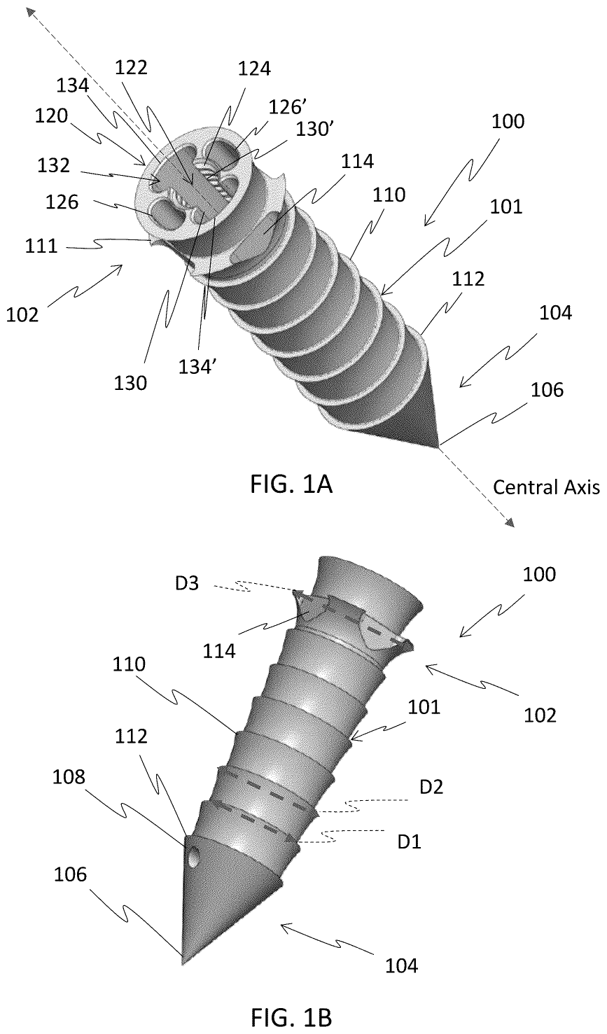

With reference now to FIGS. 1A and 1B, a suture anchor 100 is shown according to one embodiment. The suture anchor 100 has an elongate anchor body 101 having a proximal end 102 and a distal end 104. An anchoring element such as a barb 110 is protruding from the elongate anchor body 101 to secure the anchor 100 into bone. The anchoring element can also be for example one or more threads or a combination or major and minor threads or barbs. The anchor material can be for example a material known in the art, including a medical grade polymer such as PEEK, or a medical grade metal such as titanium or stainless steel.

Openings are configured in a proximal face 120 of the elongate anchor body 101, which can include a central eyelet shuttle opening 122 and at least one driver opening 126, which in FIGS. 1A and 1B is embodied as a pair of driver openings 126, 126′. The pair of driver openings 126, 126′ can be configured on opposing sides of the central eyelet shuttle opening 122. The central eyelet shuttle opening 122 is configured between the pair of driver openings 126, 126′. The central eyelet shuttle opening 122 is configured along a central axis of the elongate anchor body 101 with threads 124 for driving the eyelet shuttle along the central axis into the anchor's interior (or out the anchor's interior for purposes of suture readjustment). The openings also include at least one eyelet lock opening 134, which in the embodiment of FIGS. 1A and 1B include a pair of eyelet lock openings 134, 134′. The pair of eyelet lock openings 134, 134′ are configured on opposing sides of the central eyelet shuttle opening 122. As explained further below, the eyelet lock openings 134, 134′ interface with protrusions on the eyelet shuttle to arrest rotational movement of the eyelet shuttle. This arrested rotational movement allows the eyelet shuttle's breakaway feature to trigger, separating the eyelet shuttle's eyelet and threaded portion as the threaded portion continues to spin. The central opening 122 can be configured between the pair of eyelet lock openings 134, 134′. The central opening 122 includes at least one internal slot 130 running parallel to the central axis, which is depicted in FIGS. 1A and 1B as a pair of internal slots 130, 130′ running parallel to the central axis. The pair of internal slots 130, 130′ are configured on opposing sides of the central opening 122 and guide axial movement of the eyelet down into the anchor. The central opening 122 is configured between the pair of internal slots 130, 130′ and along with the pair of eyelet lock openings 134, 134′ define a common proximal void 132. The common proximal void 132 can have a perimeter with at least four convex regions.

The distal end 104 has a pointed distal tip 106. A distal eyelet 108 is configured between the distal tip 106 and a first distal barb 112 which extends laterally though the elongate body 101. Most of the barbs 110 have a geometry that tapers away from a first diameter D1 (sometimes referred to as the “root” of the anchor) to a larger second diameter D2. A proximal barb 111 can include multiple recesses 114 and have a larger diameter D3 than distal barbs of diameter D2. This proximal barb 111 geometry facilitates final anchoring. The multiple recesses 114 give the larger proximal barb 111 more play during insertion, given its larger diameter. The recesses can be configured as four equally spaced apart recesses.

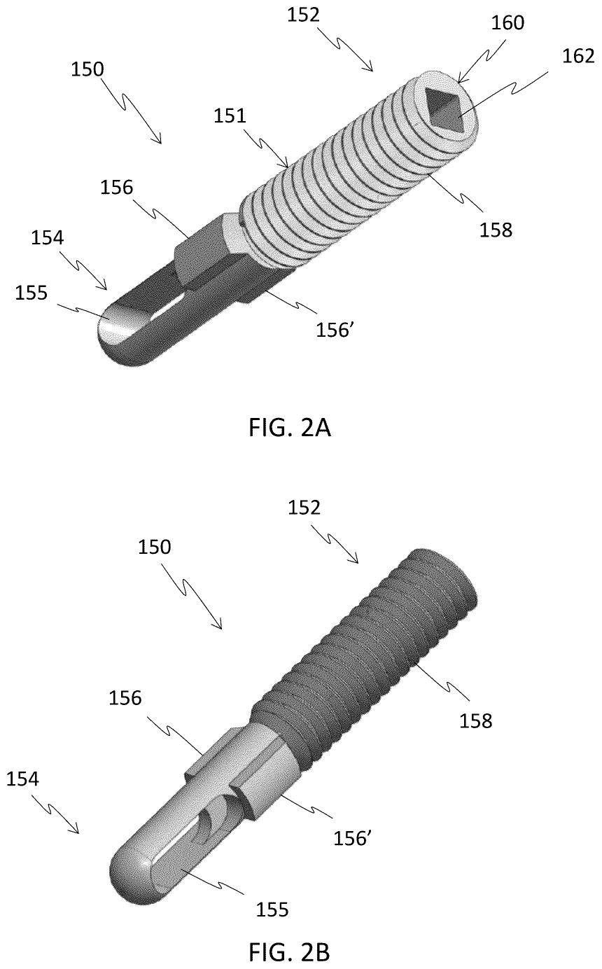

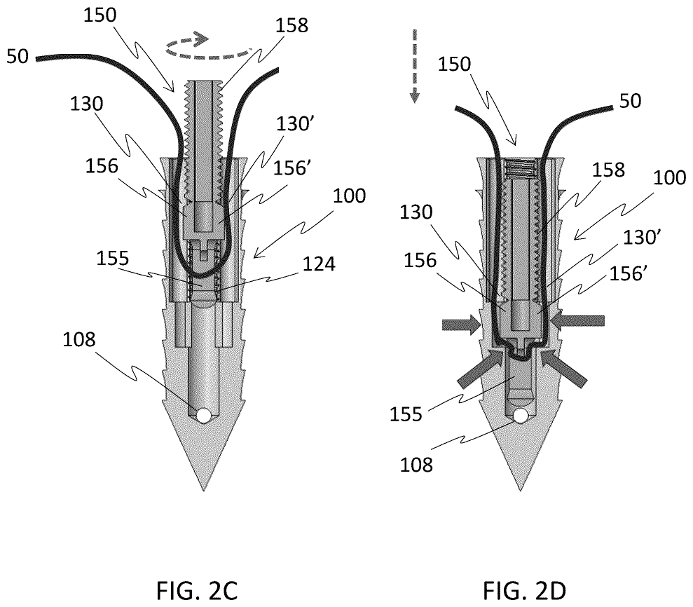

With reference now to FIGS. 2A and 2B, an eyelet shuttle 150 is shown according to one embodiment. The eyelet shuttle 150 has an elongate shuttle body 151 having a proximal end 152 and distal end 154. An eyelet 155 and at least one eyelet lock 156, depicted in FIGS. 2A and 2B as a pair of eyelet locks 156, 156′, are positioned distal of the threaded portion 158. A proximal shuttle opening 162 is disposed in a proximal face 160 of the elongate shuttle body 151. The distal eyelet 155 and eyelet locks 156, 156′ are distal of the threaded portion 158. The distal eyelet 155 can be configured distal of the eyelet locks 156, 156′. With reference now to FIGS. 2C and 2D, the at least one eyelet lock 156 is configured as a protrusion to arrest rotational movement of the eyelet 155 while the threaded portion 158 is rotating. This opposing motion provides a breakaway feature that separates the eyelet 155 and threaded portion 158 as the threaded portion 158 is rotating and the eyelet 155 is arrested. The eyelet locks 156, 156′ allow axial movement of the eyelet 155 along the central axis of the anchor 100 while the threaded portion 158 is rotating and moving distally into the anchor's interior via the threads 124. This axial movement initiated by rotation of the threaded portion 158 (e.g. clockwise rotation) pushes the eyelet 155 carrying sutures 50 distally, pulling the sutures 50 down into the anchor 100 and holding the eyelet 155 in place while compressing or pinching sutures 50 against an interior of the anchor body 101 (see e.g. arrows, FIG. 2D) to lock the suture in place. The locking friction can be achieved by compressive forces exerted on the sutures 50 by one or more vertical and horizontal walls of the eyelet 155. In addition to axial compression, radial compression can be applied as the eyelet locks 156, 156′ move into narrowing internal slots 130, 130′ within the anchor body 101.

Advantageously, the reverse movement of the threaded portion 158 (e.g. counter clockwise rotation) loosens the eyelet's 155 position distally, allowing readjustment of the sutures 50. The ability to lock, unlock and adjust tension on sutures multiple times before concluding the surgical procedure provides a major advantage.

A flexible tab or protrusion can be configured to create an audible click triggered by movement of the eyelet 155 or threaded portion 158 in an interior of the anchor, indicating to the surgeon they've sufficiently tightened and locked the sutures in place. Placement of the flexible tab or protrusion can correspond with the point the eyelet 155 or threaded portion 158 are sufficiently distal to lock the sutures. The pair of eyelet locks 156, 156′ are sized as a set of opposing protrusions and configured to be slidable within the eyelet lock openings 134, 134′ of the anchor 100.

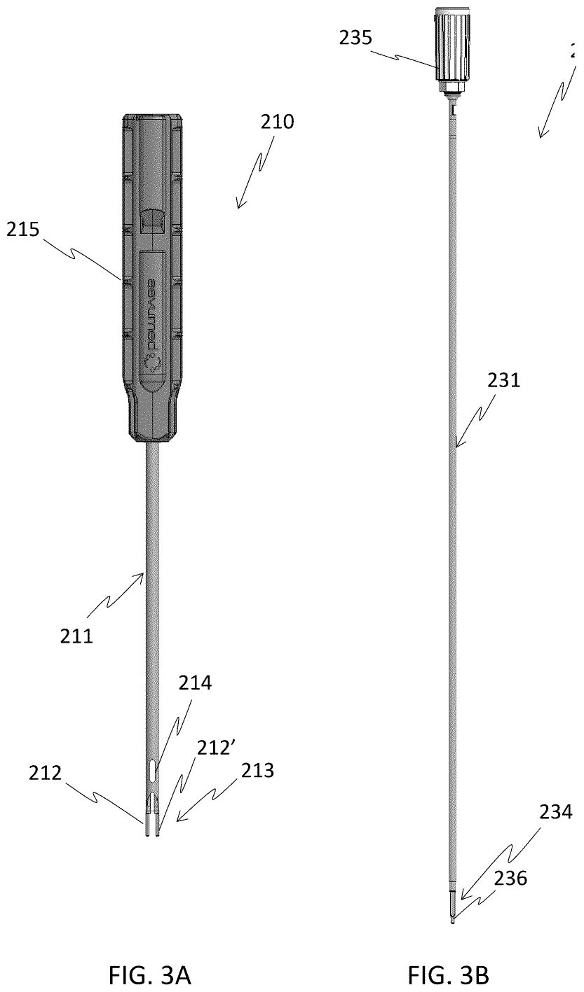

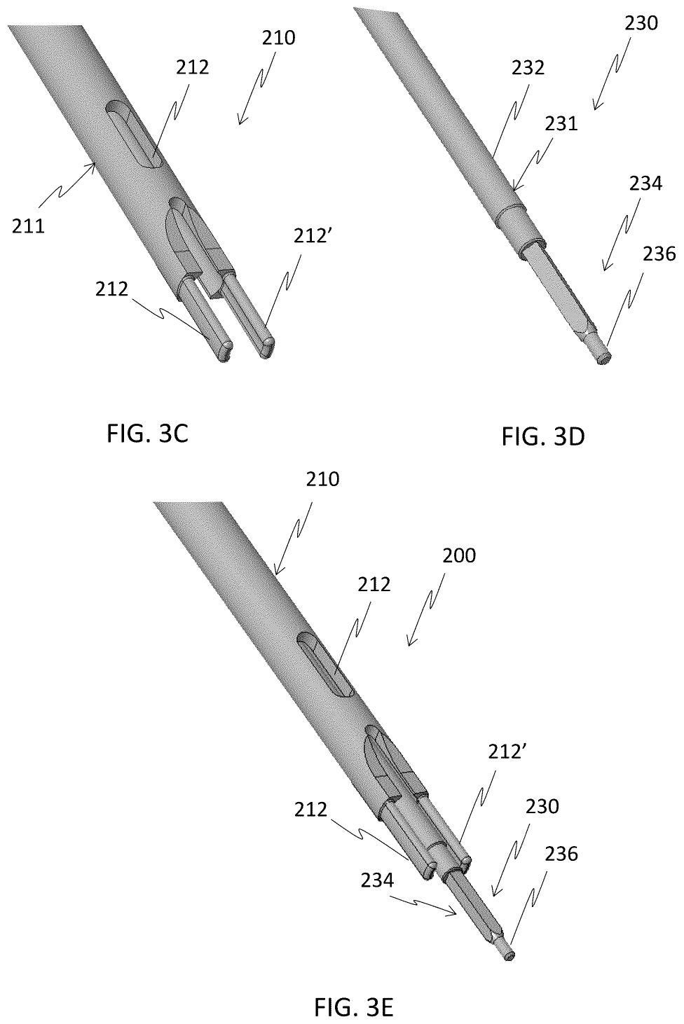

With reference now to FIGS. 3A-3E, a driver system 200 is shown according to one embodiment. The driver system 200 includes an outer driver 210 having a tubular elongate outer driver body 211 connected to an outer driver handle 215 at the proximal end, and an outer driver distal tip 213 configured to interface with driver opening on the anchor 100, which here is shown as a pair of prongs 212, 212′ configured to interface with the pair of driver openings 126, 126′. A window 214 is configured in the outer driver body 211 proximal of the outer driver distal tip 213. The elongate outer driver body 211 is configured to accept insertion of the elongate inner driver body 231 at least partially therethrough. The inner driver 230 in one embodiment has an elongate inner driver body 231 connected to an inner driver handle 235 at the proximal end, and an inner driver distal portion 234 is configured to interface with the shuttle opening 162. The inner driver distal portion 234 can have a non-circular cross-sectional profile portion (e.g. a square or rectangular profile) that terminates distally into a circular distal tip 236.

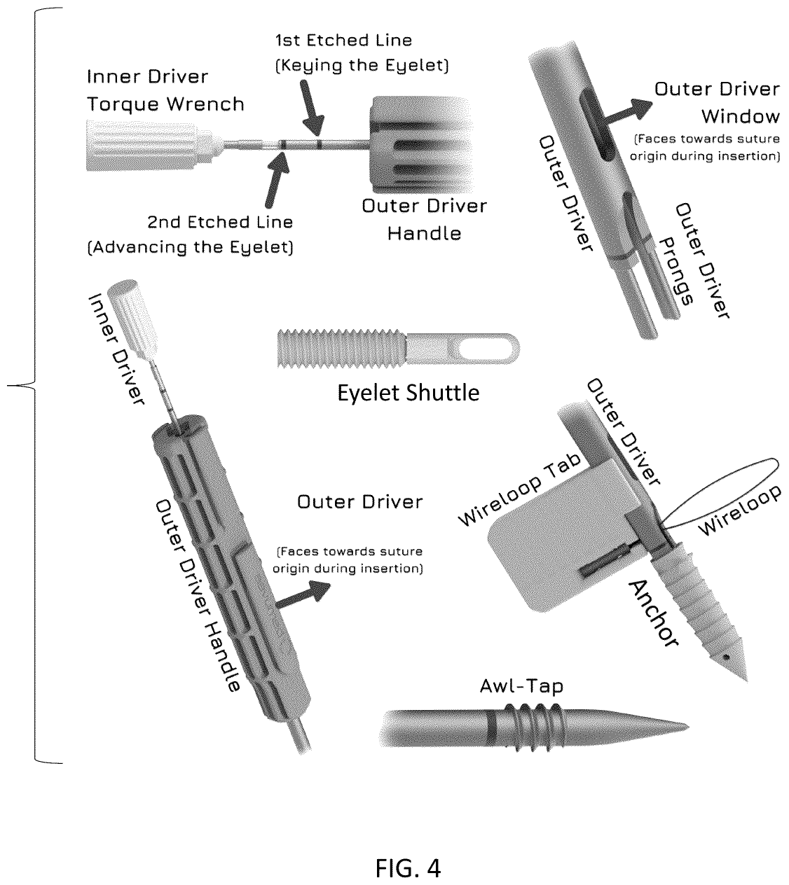

As shown in the kit view of FIG. 4, according to one embodiment, a wireloop tab can be included. The wireloop tap has a wireloop connected to a tap having at least one surface configured to interface with a surface of the outer driver. This facilitates easier loading of sutures into the eyelet. Other components such as an awl-tap can be included.

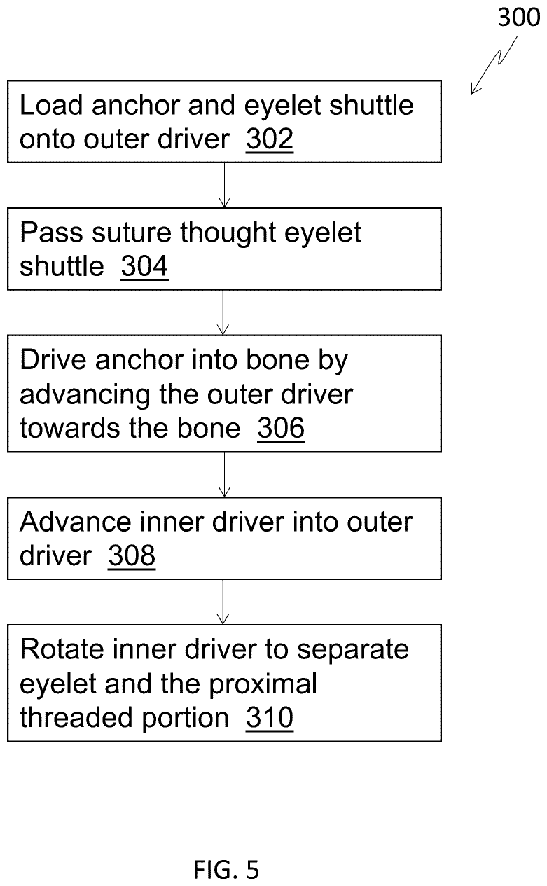

A method for inserting a suture anchor 300 will now be described with reference to FIG. 5, according to one embodiment. First, an anchor and an eyelet shuttle are loaded onto an outer driver, the eyelet shuttle having an eyelet distal of a proximal threaded portion 302. At least one suture is passed thought the eyelet shuttle 304. The anchor is driven into bone by advancing the outer driver towards the bone 306. The inner driver is then advanced into the outer driver 308, and the inner driver is twisted to separate the eyelet and the proximal threaded portion 310. Twisting the inner driver moves the threaded eyelet axially in a distal direction within the anchor. For example, twisting the inner driver clockwise can move the eyelet distally to pull down the sutures and lock them in place against a pinch or compression point. Twisting the inner driver counter clockwise can relieve this distal force on the eyelet and sutures, allowing for readjustment of tension before locking again.

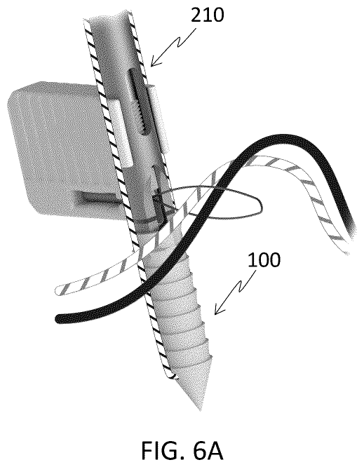

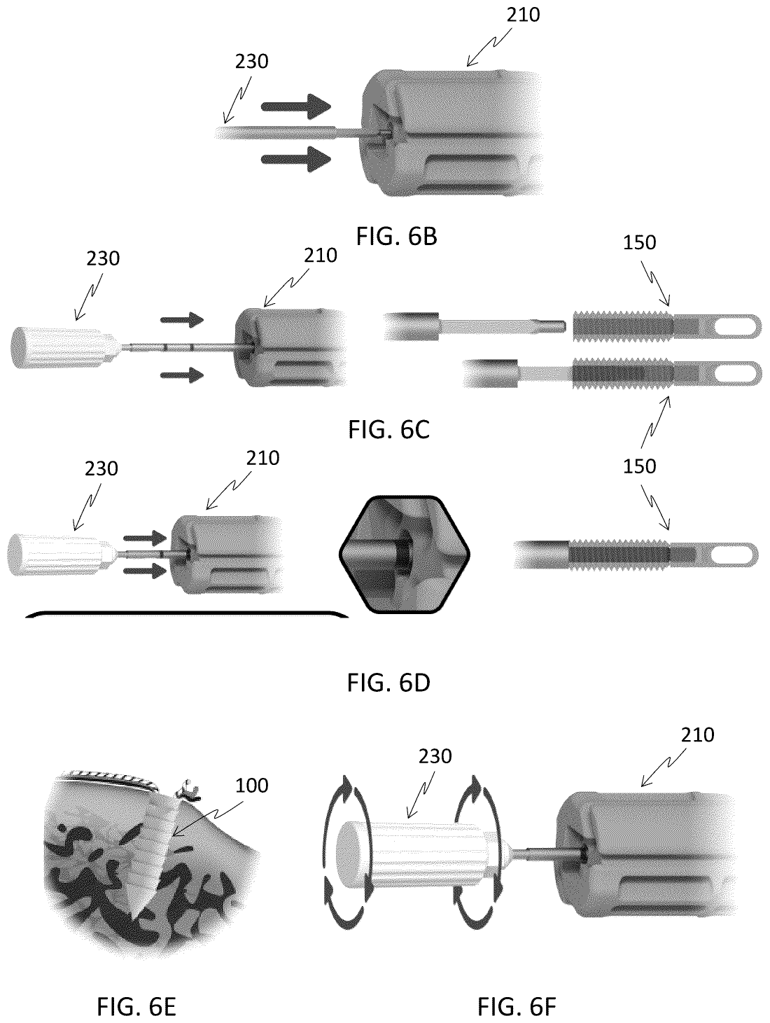

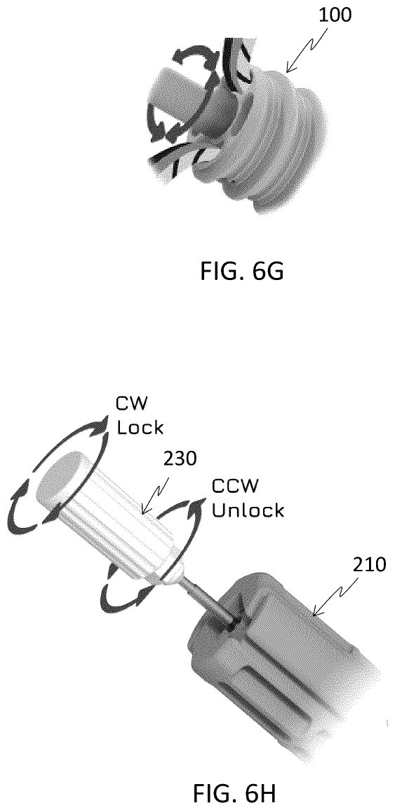

A method for implanting a suture anchor will now be described in more detail, with reference to FIGS. 6A-6H according to one embodiment. With reference to FIG. 6A, sutures are passed through the eyelet. Sutures can pass for example from a tendon though the wire loop attached to the distal eyelet. Then the wire loop tab can be pulled through while taking some of the slack out of the sutures, while also maintaining the sutures coming from the medial/superior through to inferior into the cannula. The surgeon can maintain light suture tension through the cannula until the tip of the anchor reaches the bone surface. The surgeon may impact the inserter a few times to assess the anchor's penetration into bone. If the bone density is appropriate, the window on the driver can be oriented so that the driver on the handle is towards the suture origin. Then the surgeon can proceed to inserting the anchor to the black etched line. If bone density is not appropriate, the owl tap from the kit is available.

With reference now to FIG. 6B, once the anchor is implanted, the surgeon may slowly insert the inner driver into the outer driver. With reference now to FIG. 6C, the surgeon may lightly push the inner driver like a key to feel the square head tip of the inner driver drop into the square of the eyelet screw. A quarter turn of the inner driver may be required for the drop to be felt. With reference now to FIG. 6D, once the drop is felt, the surgeon should lightly press using the thumb and index finger with no tension on the sutures until the first etched line is reached. With reference now to FIG. 6E, the sutures are now self-contained within the anchor to prevent sutures from cutting though bone under tension. With reference now to FIG. 6F, the eyelet can be advanced to the second etched line. The surgeon should turn the inner driver without tension on the sutures, towards the second etched line to engage the self-cleaning feature. At this point the suture should not cleat to the handle. There will be enough friction in the system to prevent the sutures from backing out, but not so much that the sutures cannot be tensioned.

The self-cleating feature can now be utilized. The surgeon can pull on each suture for optimized tension. Once this has occurred, the surgeon can then proceed to lock the sutures in place by continuing to twist until an audible click sound is heard. This will indicate the sutures are now locked. The surgeon may opt not to cut the sutures at this point to allow for further adjustment to the total construct if needed after additional lateral anchors are placed. With reference now to FIG. 6G, the ability to lock and unlock the sutures multiple times for optimal tensioning provides a major advantage as described above. With reference now to FIG. 6H, if adjustment to the construct is needed, unlock the sutures by turning the inner driver handle counterclockwise approximately 6 rotations. Then tension each suture individually as desired and relax sutures again by turning the inner driver clockwise until an audible sound is heard. The surgeon may then cut sutures as appropriate after final adjustments. At this point, the retaining suture seated between the anchor and the bone can now be cut as well.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention.

What is claimed is:

1. A suture anchor system comprising:
a suture anchor comprising:
an elongate anchor body having at least one anchoring element, and
a plurality of openings in a proximal face of the elongate anchor body; and
an eyelet shuttle comprising:
an elongate shuttle body having an eyelet and at least one eyelet lock distal of a proximal threaded portion, and a proximal shuttle opening in a proximal face of the elongate shuttle body.

2. The system of claim 1, wherein the plurality of openings comprises a central opening and at least one driver opening.

3. The system of claim 2, wherein the at least one driver opening is one of a pair of driver openings.

4. The system of claim 3, wherein the pair of driver openings are configured on opposing sides of the central opening.

5. The system of claim 3, wherein the central opening is configured between the pair of driver openings.

6. The system of claim 3, wherein the central opening is configured along a central axis of the elongate anchor body.

7. The system of claim 3, wherein the plurality of openings comprises at least one eyelet lock opening.

8. The system of claim 7, wherein the eyelet lock opening is one of a pair of eyelet lock openings.

9. The system of claim 8, wherein the pair of eyelet lock openings are configured on opposing sides of the central opening.

10. The system of claim 8, wherein a central opening is configured between the pair of eyelet lock openings.

11. The system of claim 8, wherein the central opening includes at least one internal slot running parallel to the central axis.

12. The system of claim 11, wherein the at least one internal slot is one of a pair of internal slots running parallel to the central axis.

13. The system of claim 12, wherein the pair of internal slots are configured on opposing sides of the central opening.

14. The system of claim 12, wherein the central opening is configured between the pair of internal slots and along with the pair of eyelet lock openings define a common proximal void.

15. The system of claim 14, wherein the common proximal void comprises a perimeter having at least four convex regions.

16. The system of claim 1, wherein the distal end comprises a pointed distal tip.

17. The system of claim 16, wherein a distal eyelet is configured between the distal tip and a first distal barb.

18. The system of claim 1, wherein the at least one anchoring element comprises a plurality of barbs.

19. The system of claim 18, wherein each of the plurality of barbs tapers away from a first diameter to a second diameter.

20. The system of claim 19 further comprising:
a proximal barb comprising a plurality of recesses.

21. The system of claim 20, wherein the plurality of recesses is four recesses equally spaced apart.

22. The system of claim 20, wherein the proximal barb tapers away from a first diameter to a third diameter, wherein the third diameter is greater than the second diameter.

23. The system of claim 1, wherein the at least one eyelet lock is configured to arrest rotational movement of the eyelet while the threaded portion is rotating to separate the eyelet and threaded portion.

24. The system of claim 1, wherein the at least one eyelet lock is configured to allow axial movement of the eyelet within the anchor while the threaded portion is rotating and while the eyelet and threaded portion are separated.

25. The system of claim 1, wherein the at least one eyelet lock is one of a pair of eyelet locks.

26. The system of claim 25, wherein the pair of eyelet locks are a set of opposing protrusions.

27. The system of claim 1 further comprising:
an inner driver having an elongate inner driver body and an inner driver distal portion configured to interface with the shuttle opening; and
an outer driver having an elongate outer driver body an outer driver distal tip configured to interface with a pair of driver openings in a proximal face of the elongate anchor body, the elongate outer driver body configured to accept insertion of the elongate inner driver body at least partially therethrough.

28. The system of claim 27, wherein the outer driver distal tip comprises a pair of prongs configured to interface with a pair of driver openings.

29. The system of claim 27, wherein the inner driver distal portion has a non-circular cross-sectional profile portion.

30. The system of claim 27, wherein the inner driver distal portion has a non-circular cross-sectional profile portion that terminates into a circular distal tip.

31. The system of claim 27 further comprising:
a wireloop tab comprising a wireloop connected to a tap having at least one surface configured to interface with a surface of the outer driver.

32. A method for inserting a suture anchor comprising:
loading an anchor and an eyelet shuttle onto an outer driver, the eyelet shuttle comprising an eyelet distal of a proximal threaded portion;
passing at least one suture thought the eyelet shuttle;
driving the anchor into bone by advancing the outer driver towards the bone;
advancing an inner driver into the outer driver; and
rotating the inner driver to separate the eyelet and the proximal threaded portion.

33. The method of claim 32, wherein rotating the inner driver moves the eyelet axially within the anchor.

34. The method of claim 32, wherein rotating the inner driver in a first direction locks the at least one suture.

35. The method of claim 32, wherein rotating the inner driver in a second direction opposite the first direction unlocks the at least one suture.
